(19) United States
(12) Patent Application Publication
(10) Pub. No.: US 2017/0342373 A1
(43) Pub. Date: Nov. 30, 2017

(54) TECHNIQUE FOR FORMATION AND ASSEMBLY OF 3D CELLULAR STRUCTURES

(71) Applicant: Agency for Science, Technology and Research, Singapore (SG)

(72) Inventors: Andrew Chwee Aun WAN, Singapore (SG); Meng Fatt LEONG, Singapore (SG); Tze Chiun LIM, Singapore (SG); Hong Fang LU, Singapore (SG)

(21) Appl. No.: 15/540,756

(22) PCT Filed: Dec. 30, 2015
(86) PCT No.: PCT/SG2015/050516

§ 371 Date: Jun. 29, 2017

(30) Foreign Application Priority Data
Dec. 30, 2014 (SG) 10201408826X

(51) Int. Cl. C12N 5/00; A61K 35/12; A61K 35/00

(52) U.S. Cl. CPC C12N 5/0062; A61K 35/12; A61K 35/00; C12N 5/00

(57) ABSTRACT

In one example, the present invention refers to a method of making or producing a self-supporting cellular construct having a continuous channel within its central cavity, comprising the steps of providing a mould with a central opening, wherein the mould encloses a volume around the central opening (hole) capable of housing a plurality of cells. Each of the plurality of self-supporting cellular constructs, having a central opening in a series adjacent to one another such, is placed so that the central opening of each of the self-supporting cellular constructs having a central opening is aligned to one another to thereby form, for example, a continuous channel within its central cavity.

### CROSS-REFERENCE TO RELATED APPLICATIONS

This application claims the benefit of priority of Singapore provisional application No. 10201408826X, filed 31 Dec. 2014, the contents of it being hereby incorporated by reference in its entirety for all purposes.

### FIELD OF THE INVENTION

The present invention relates generally to the field of tissue engineering. In particular, the present invention relates to the formation and assembly of 3D cellular structures.

### BACKGROUND OF THE INVENTION

Tissue engineering aims to create functional cellular constructs to replace damaged tissues or organs. To create tissue constructs, cells are grown on scaffolds or in gels that contain biochemical and physical cues to organize cells three dimensionally (3D). As a result, tissue constructs created using existing 3D cellular organization techniques often contain scaffold or gel materials. However, before these 3D tissue constructs can be implanted, issues such as the material biocompatibility (before and after degradation), ability of the material to support vascularization and stability of the 3D structure after material degradation need to be addressed.

There is therefore a need to provide alternative constructs and methods for producing 3D tissue constructs obviating one or more of the above disadvantages.

### SUMMARY OF THE INVENTION

In one aspect, the present invention refers to a method of making a self-supporting cellular construct having a continuous channel within its central cavity, wherein the method comprises the steps of providing a mould with a central opening, wherein the mould encloses a volume around the central opening (hole) capable of housing a plurality of cells; inserting a plurality of cells in a cell culture medium into the mould; allowing the cells to grow and form a self-supporting cellular construct within the mould; removing the self-supporting cellular construct having a central opening and an outer dimension of the mould from the mould; repeating the steps as described above to obtain a plurality of self-supporting cellular construct having a central opening; placing each of the plurality of self-supporting cellular constructs having a central opening in a series adjacent to one another such that the central opening of each of the self-supporting cellular constructs having a central opening is aligned to one another to thereby form a continuous channel within its central cavity.

In another aspect, the present invention refers to a method of making a self-supporting cellular construct, the method comprises the steps of providing a mould of a desired shape, wherein the mould comprises an area capable of housing a plurality of cells and a removable material within the mould; inserting a plurality of cells in a cell culture medium into the mould; inserting a removable material before or after the previous step; allowing the cells to grow and form a self-supporting cellular structure surrounding the removable material within the mould; and removing the self-supporting cellular structure from the mould to thereby form a self-supporting cellular construct.

In yet another aspect, the present invention refers to a material-free cellular construct as described herein.

In a further aspect, the present invention refers to a material-free tubular cellular construct as described herein.

In another aspect, the present invention refers to a method of testing chemical agent comprising the steps of providing the cellular construct as described herein; and providing the chemical agent through the channel of the cellular construct and observing changes to the plurality of cells of the cellular constructs.

### BRIEF DESCRIPTION OF THE DRAWINGS

The invention will be better understood with reference to the detailed description when considered in conjunction with the non-limiting examples and the accompanying drawings, in which:

FIG. 1 shows an assembly of cellular units used to form 3D cellular structures. (a)-(c) are images showing the formation of spheroids consisting of a mixture of hepatocarcinoma cells (hepG2) and endothelial cells (EndoGFP). (d)-(f) are images showing the formation of endothelial tube on a biodegradable suture in a mould, such as a PDMS mould. (g)-(i) are images showing the assembly of the cellular spheroids on an endothelial tube to form higher order tissue structure. (a), (d) and (g) are schematic diagrams, whereby (a) shows spheroids formed from cells. (d) shows three different types of tubes. The tube in the top left of the schematic is a tube of cellular material (dark grey) around a rod of removable matter (light grey). The middle tube shows a cross-section of the tube of cellular material, whereby the removable material is being extracted from the tube. The tube on the bottom right shows the resulting cellular tube after extraction of the removable material. The remaining images are micrograph images.

FIG. 2 shows the formation of cellular spheroids using a micro-well mould. (a) is a schematic representation of the process of obtaining uniform cellular spheroids using moulds, for example a multi-welled, PDMS micro-well mould. (b) is a micrograph of (EndoGFP) cells packed into the micro-wells by centrifugation. (c) is a micrograph showing (EndoGFP) cells aggregated inside micro-wells after 1 day in culture. (d) is a micrograph showing the uniform-sized cellular spheroids obtained by rinsing them off the micro-well mould.

FIG. 3 shows micrograph images and column charts visualising the relationship between spheroid size and cell number. The micrograph images (a)-(d) show aggregates formed by seeding 0.5, 1, 2 and 3 million EndoGFP cells, respectively. The column graph (e) shows an increase in aggregate size which is concurrent with an increase in cell number for single cell type (EndoGFP) and mixed cell type (EndoGFP:HepG2, mixing ratio 1:1). Column graph (f) shows an increase in aggregate size concurrently with an increase in micro-well size.

FIG. 4 is a schematic showing the formation of cellular spheroids on sutures. In this schematic, rods or sutures are laid across the micro-well mould, thereby enabling cellular spheroids to loosely attach to the suture. The position of the spheroids may be gathered or removed from the suture by gently pushing the attached spheroids in the desired direction. Micrograph images of this schematic are shown in FIG. 5 below.

FIG. 5 are micrograph images showing the formation of patterned cellular unit on a suture. A schematic of these images is provided in FIG. 4 above. Images (a)-(b) show individual spheroids of different cell types that formed on the suture after 24 hours. Images (c)-(d) show spheroids when brought into contact with each other and images (e)-(f) show spheroids fused into a patterned cellular unit after further culturing. (a), (c) and (e) are light micrographs, while (b), (d) and (f) are the corresponding fluorescence micrographs, respectively.

FIG. 6 depicts the formation of cellular tubes on a suture in a mould. Image (a) is a schematic diagram showing the process of obtaining cellular tubes using a tube mould, such as a PDMS tube mould. Sutures are laid across tube moulds and cells deposited into the tube moulds condense into tubes and attach loosely to the sutures. These tubes can then be removed from the mould with the help of the sutures. These sutures can also be further removed, resulting in the formation of an annular, cellular tube. Image (b) shows EndoGFP cells packed into the tubular channel by centrifugation. (c) shows EndoGFP cells aggregated around the suture inside the channel after 1 day. (d) shows that the cellular tube can be removed with the suture from the mould.

FIG. 7 shows micrograph images and column graphs representing the relationship between cellular tube thickness and cell number. (a)-(d) are micrograph images of cellular tubes formed by seeding 0.5, 1, 2 and 3 million EndoGFP cells respectively. (e) is a column graph showing an increase in cellular tube outer diameter with an increase in cell number.

FIG. 8 comprises micrograph images of the formation of cellular tubes with primary HUVEC cells. (a) shows a mixture of HUVEC and hSMC (ratio 4:1), which were seeded into tubular channel. (b) and (c) show cells that condensed onto suture after 1 day in culture.

FIG. 9 shows micrograph images showing the cell viability assays performed on HUVEC cellular tubes at day 7. (a) is a Bright field image, (b) shows live cells (originally stained with calcein AM), (c) shows dead cells (originally stained with ethidium homodimer, (d) shows the merged image of (b) and (c).

FIG. 10 shows micrograph images showing the tubulogenesis assay of HUVEC tubes in collagen gel at day 7.

FIG. 11 shows micrograph images of immunostaining of HUVEC tube performed on day 7 for collagen I and CD31. Nuclei were stained with DAPI. (a) and (b) are longitudinal sections, (c) and (d) are cross sections. Scale bars: 100 μm.

FIG. 12 shows images of the immunostaining of HUVEC construct at day 7 for basement membrane extracellular matrix (ECM) components. (a)-(c) show the staining results for Collagen IV and CD31. (d)-(f) show the staining results for laminin and CD31. Nuclei were stained with DAPI. Positive immunostaining of cells for Collagen I, Collagen IV and laminin indicate a robust production of extracellular matrix by the cells, and networks of endothelial cells within the construct.

FIG. 13 shows the formation of tissue construct with interfacial polyelectrolyte (IPC) fibres. Smooth muscle cells (SMC)-laden IPC fibres were wrapped circumferentially around cellular tube formed by EndoGFP cells. Images (a)-(b) show the construct at a low magnification and (c)-(d) are high magnification images. (a) and (c) are light micrographs, while (b) and (d) are the corresponding fluorescence micrographs, respectively.

FIG. 14 is a schematic diagram showing the arrangement of cellular units into more complex structures. In this diagram, spheroids attached to sutures are assembled around a cellular tube in the middle of the assembly. This is formed by using cellular tubes as shown in FIG. 6 and suture-attached spheroids as shown in FIG. 5. The schematic of FIG. 14 can be considered a variation of the complex structure shown in FIG. 1(g) to (i).

FIG. 15 shows the assembly of cellular rings to form perfusable cellular tube. (a)-(d) are micrograph images showing the formation of cellular rings using, for example, a PDMS mould. (a) shows an empty mould. (b) shows a mould containing a suspension of seeded cells. (c) shows cells condensed around the mould after 1-2 days. (e)-(f) are schematic diagrams showing the assembly of cellular rings around a rod, which could later be removed to form a hollow cellular tube. (g) is a micrograph image showing cellular rings fused to form a cellular tube on a rod. This image corresponds to the schematic diagram found in (e). (h) is a micrograph image showing that upon removal of the rod, the hollow cellular tube could be perfused through its lumen. Scale bars: 500 μm. (i) is the same image as image (e), providing more detailed information.

FIG. 16 shows a schematic representation of an example of a perfusable vascular network, showing exemplary cell types and functions.

### DEFINITIONS

As used herein, the term “friction” refers to the force resisting the relative motion of solid surfaces, fluid layers, and material elements sliding against each other. There are several types of friction known in the art, for example dry friction (the relative lateral motion of two solid surfaces in contact, static friction (between non-moving surfaces), kinetic friction (between moving surfaces), fluid friction (friction between layers of a viscous fluid that are moving relative to each other), lubricated friction (fluid friction where a lubricant fluid separates two solid surfaces), skin friction (a component of drag, the force resisting the motion of a fluid across the surface of a body) and internal friction (the force resisting motion between the elements making up a solid material while it undergoes deformation). When surfaces in contact move relative to each other, the friction between the two surfaces converts kinetic energy into thermal energy, meaning that work energy is converted to heat. This property can have dramatic consequences on the surfaces in question, for example in the use of friction created by rubbing pieces of wood together to start a fire. Kinetic energy is converted to thermal energy whenever motion with friction occurs, for example when a viscous fluid is stirred. Another important consequence of many types of friction can be wear, which may lead to performance degradation and/or damage to components exposed to friction.

As used herein, the term “3D” or “three-dimensional” refers to a measurement in space. One dimensional (1D) refers to a measurement in any one direction of the three spatial directions; specifically, one of three coordinates determining a position in space. Two dimensional (2D) therefore refers to a measurement in two of the three spatial directions. If information is provided pertaining to all three spatial coordinates, this information is then understood to describe a three-dimensional object.

As used herein, the term “non-stick” refers to a surface engineered to reduce the ability of other materials to stick to it or adhere to it. An agent that has this characteristic is referred to as a non-stick agent. The main characteristic of non-stick agents is their low coefficient of friction on their surfaces, thereby resulting in the non-sticking effect when moved or placed against other surfaces. As further known in the art, non-sticking agents are usually hydrophobic and demonstrate a high electron negativity.

As used herein, the term “mould” refers to a hollow container with a particular, defined shape into which substances (mostly soft, liquid or malleable) are poured or placed, so that when the substance becomes hard it takes the shape of the container. In the present disclosure, the term “mould” refers to a hollow container that is usable for cell culture techniques, whereby cells that are placed into the mould are capable of further forming adhesions, proliferating and/or differentiating, in accordance to standard cell culture techniques.

As used herein, the term “self-supporting cellular construct” refers to the capability of a cellular structure to maintain its structure without the assistance of any external material.

As used herein, the term “cell culture” refers to the removal of cells from an animal or plant and their subsequent growth in a favourable artificial environment. The cells may be removed from the tissue directly and disaggregated by enzymatic or mechanical means before cultivation, or they may be derived from a cell line or cell strain that has already been established. Primary culture refers to the stage of the culture after the cells are isolated from the tissue and proliferated under the appropriate conditions until they occupy all of the available substrate (i.e., reach confluence). At this stage, the cells have to be sub-cultured (i.e., passaged) by transferring them to a new vessel with fresh growth medium to provide more room for continued growth. After the first subculture, the primary culture becomes known as a cell line or sub-clone. Cell lines derived from primary cultures have a limited life span (i.e., they are finite), and as they are passaged, cells with the highest growth capacity predominate, resulting in a degree of genotypic and phenotypic uniformity in the population. If a subpopulation of a cell line is positively selected from the culture by cloning or some other method, this cell line becomes a cell strain. A cell strain often acquires additional genetic changes subsequent to the initiation of the parent line. Normal cells usually divide only a limited number of times before losing their ability to proliferate, which is a genetically determined event known as senescence; these cell lines are known as finite. However, some cell lines become immortal through a process called transformation, which can occur spontaneously or can be chemically or virally induced. When a finite cell line undergoes transformation and acquires the ability to divide indefinitely, it becomes a continuous cell line. Culture conditions vary widely for each cell type, but the artificial environment in which the cells are cultured invariably consists of a suitable vessel containing, but not limited to, a substrate or medium that supplies the essential nutrients (amino acids, carbohydrates, vitamins, minerals), growth factors, hormones, gases (O₂, CO₂), and a regulated physico-chemical environment (pH, osmotic pressure, temperature). Most cells are anchorage-dependent and must be cultured while attached to a solid or semi-solid substrate (adherent or monolayer culture), while others can be grown floating in the culture medium (suspension culture).

As used herein, the term “media” or “medium” refers to cell culture media, which is a chemically defined growth medium suitable for the in vitro cell culture of human or animal cells, in which all of the chemical components are known.

As used herein, the term “removable material” refers to removable scaffolding or stabilising materials used in the production of tissue and cellular constructs.

As used herein, the term “ECM” refers to the extracellular matrix. The extracellular matrix is the viscous, watery fluid that surrounds cells in animal tissue. Secreted by the cells themselves, it is the medium thought with they receive materials (e.g. nutrients, hormones) from elsewhere in the body and via which they communicate with other cells. The extracellular matrix (ECM) is the environment in which cells migrate during tissue development and it contains constituents that bind cells together to maintain tissue integrity. The bulk of the matrix consists of proteoglycans, which associate with water molecules. Other key constituents are collagens, insoluble fibre proteins that form various bundles, chains and other structural components. Also present are multi-adhesive proteins, which bind to other matrix components and to cell adhesion molecules in plasma membranes. The extracellular matrix (ECM) is especially prominent in connective tissues, such as bone, cartilage, and adipose tissue, in which it is sometimes called ground substance.

As used herein, the term “material-free” refers to the characteristic of a cellular construct to be free of foreign material. Usually, supporting or scaffolding material is used to provide initial stability when cultivating tissue constructs in vitro. Complete removal of this scaffolding material prior to implantation of the cellular/tissue construct renders the construct “material-free”.

As used herein, the term “parenchyma” or “parenchymal cells”, in animals, are the functional parts of an organ in the body. This is in contrast to the stroma, which refers to the structural tissue of organs, namely, the connective tissues. Examples for parenchyma in humans are, but not limited to, neurons and glia cells in the brain, myocytes in the heart, nephrons in the kidneys, hepatocytes in the liver, alveolar tissue in the lungs, white and red pulp in the spleen, follicles in the ovaries, and Langerhans cells in the pancreas.

As used herein, the term “adherent cells” refers to mammalian cells that grow attached to a surface, for example the bottom of a cell-culture dish. These cell types have to be detached from the surfaces to which they adhere before they can be passaged or sub-cultured. For adherent cells, cell density is normally measured in terms of confluency, which is the percentage of the surface covered by cell growth (cell density). The cells will often have a preferred range of confluencies for optimal growth. For example, mammalian cell lines, such as HeLa or Raw 264.7, generally prefer confluencies over 10% but under 100%. When passaging these cells one normally tries to keep the cell density in this range. When passaging, cells may be detached by one of several methods known in the art, including but not limited to, trypsin treatment in order to break down the proteins responsible for surface adherence, Other methods include, but are not limited to chelating sodium ions with ethylenediaminetetraacetic acid (EDTA), which disrupts some protein adherence mechanisms, or mechanical methods such as repeated washing or use of a cell scraper. The detached cells are then re-suspended in fresh growth medium and allowed to settle back onto their growth surface for re-attachment. Cells that do not grown attached to surfaces are known as “suspension cells”. These cells are generally grown in solution, whereby usually the solution is in constant agitation, in order to prevent uneven accumulation of the cells in the growth media, thereby enabling constant and even growth throughout.

As used herein, the term “aggregate” refers to the ability of some cells to form intimate, condensed clusters with clear cell-cell junctions forming in between the cells. A cell can be both adherent and form aggregates at the same time.

### DETAILED DESCRIPTION OF THE PRESENT INVENTION

Provided herein is an alternate method to circumvent material issues, such as material biocompatibility (before or after degradation), the ability of the material to support vascularisation and the stability of the resulting 3D structure after material degradation. One alternative is to form cellular aggregates without the use of materials. Cells are often organized into aggregates using 3D culture techniques to promote cell-cell interactions. Improved cellular interactions have been shown to be beneficial in the preservation of pluripotency in embryonic stem cells and more recently, formation of functional organ buds or organoids. These aggregates are formed using cellular self-organization, cell sheet engineering and microgravity techniques.

When implanting cellular structures, foreign matter, such as scaffolding and other rigid materials, can be used to cultivate these cellular structures in vitro, and can thus become an issue for the transplant recipient's immune system, resulting in possibly fatal immunogenic reactions to the foreign material found in such cellular structures. Therefore, there is a need for functional cellular constructs and 3D structures that are free from foreign material, rapid in production and uniform in size for both in vivo and in vitro use.

In this disclosure, a technique to form material-free, scalable, 3D cellular units with controllable shapes and uniform size is described. These cellular units are further assembled into more complex 3D structures to resemble the complex structures of native tissues, which is important for use in organ or tissue regeneration or tissue repair. Furthermore, the present invention does not rely on other biomaterials to micropattern (which is the patterning of cells in 3D at micron resolution) or produce these cellular constructs. Instead, if at all needed, removable scaffolding and supporting materials can be used. For example, sutures can be used for assembly of complex cellular constructs, whereby the supporting sutures are later removed prior to implantation. These sutures are also available in biodegradable versions, thereby allowing the use of the suture as a physical support of the complex structure during implantation, following which the suture will dissolve over time, thereby eliminating any possible implant rejection issues due to foreign materials present in the implant (that is the self-supporting cellular construct). In terms of output and throughput, the moulds used herein can be fitted into conventional multi-welled cell culture plates or can be used in other devices suitable for culturing cells. Uniform, cellular units are formed using conventional cell culture techniques without the need for sophisticated equipment. Thus, this method can be used in a research facility or for high-throughput screening applications.

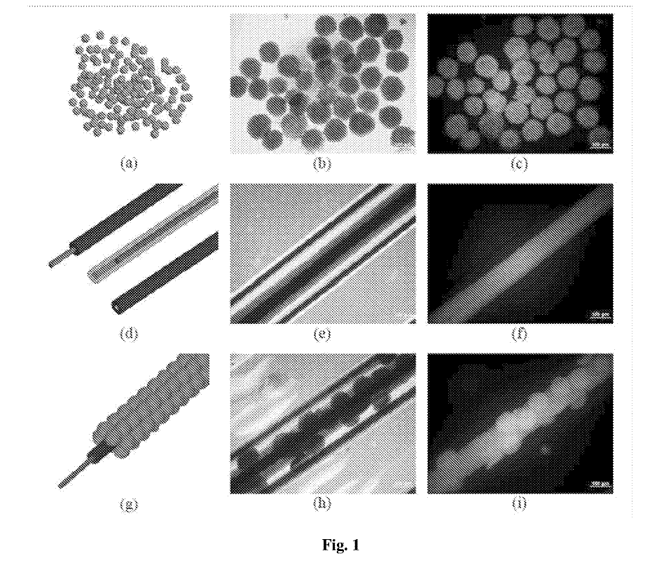

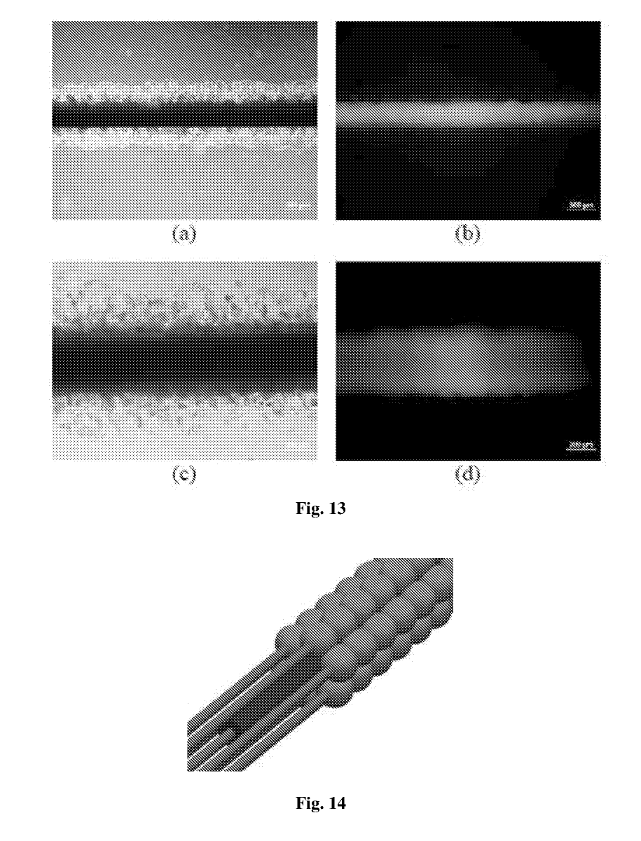

In order to assemble any complex cellular constructs, it is advantageous to initially produce small, simple structural units. For example, assembly of more complex structures is carried out by first forming desired structures (such as tubes and spheroids) separately and placing them together in a mould. (FIG. 1) The cellular structures are able to attach to each other later on in cell culture. Structures can also be formed on sutures, subsequently putting the sutures together to allow the cellular structures to fuse (FIG. 14). The size and design of the complex structure therefore dictates the size and form of the simple structural units needed to assemble the resulting complex cellular construct. A person skilled in the art, having the end result in mind, is able to devise and implement the dimensions required by the method as described herein to produce the small, simple structural units. Examples of such structural units can be seen in, for example, FIG. 1 and FIG. 5. Dividing the resulting cellular construct into smaller parts enables faster and simple production techniques, thereby improving and simplifying the work flow. These small, structural units, which themselves are also considered to be self-supporting cellular constructs, can then be assembled into larger, more complex cellular constructs, and do not pose a limitation on the size of the resulting complex cellular construct. Any limitations on the design and function of the self-supporting cellular construct would be known to a person skilled in the art. For example, it can be difficult for the resulting cellular structure to be self-supporting for structures with a diameter smaller than 50 μm. Therefore, in one example, the self-supporting cellular construct has a diameter of about 50 μm to about 10 cm or 5 cm. That is, the diameter of the self-supporting cellular construct has a diameter of about 50 μm to about 100 μm, about 100 μm to about 250 μm, about 250 μm to about 500 μm, about 500 μm (0.5 mm) to about 1000 μm (1 mm), about 1 mm to about 2 mm, about 2 mm to about 5 mm, about 5 mm to about 10 mm (1 cm), about 1 cm to about 2 cm, about 2 cm to about 5 cm, about 55 μm, about 75 μm, about 120 μm, about 150 μm, about 300 μm, about 1.5 mm, about 3.5 mm, about 7.5 mm, about 1.2 cm, about 2.5 cm, about 3.5 cm or about 4.5 cm. In one example, the cellular construct is 1 cm to 3 cm in diameter. In another example, the cellular construct is 3 cm in diameter.

In order to create self-supporting cellular constructs of those dimensions, the present invention discloses the use of a mould for forming and producing down to the smallest structural units. The size and shape of these moulds is dictated by the form and structure later required for assembling the larger, more complex constructs. These moulds are provided in multi-welled or single welled versions, depending on the size and complexity of the resulting self-supporting cellular construct. In one example, the mould is multi-welled. In one example, the mould is in any shape selected from, but not limited to, circular, elliptical, tubular (pipe-shaped), toroidal, polygonal dimensions (such as triangular, rhomboid, trapeze, pentagonal, hexagonal and the like) and of irregular shape. In one example, the mould is tubular in shape or pipe-shape structure. In yet another example, the mould is spheroidal in shape. In another example, the length of the tubular mould is about 10 μm (e.g. a few cells) to about 30 cm (for example for oesophageal tissue replacement). That means that the tubular mould has a length of about 10 μm to about 100 μm, about 100 μm to about 250 μm, about 250 μm to about 500 μm, about 500 μm (0.5 mm) to about 1000 μm (1 mm), about 1 mm to about 2 mm, about 2 mm to about 5 mm, about 5 mm to about 10 mm (1 cm), about 1 cm to about 2 cm, about 2 cm to about 5 cm, about 5 cm to about 10 cm, about 10 cm to about 15 cm, about 15 cm to about 30 cm, about 15 μm, about 35 μm, about 55 μm, about 75 μm, about 120 μm, about 150 μm, about 300 μm, about 1.5 mm, about 3.5 mm, about 7.5 mm, about 1.2 cm, about 2.5 cm, about 3.5 cm, about 4.5 cm, about 8 cm, about 12.5 cm, about 18 cm, about 25 cm or about 28 cm.

In another example, the mould is from about 10 μm to about 10 cm or 5 cm in diameter. That means that the mould has a diameter of about 10 μm to about 50 μm, about 50 μm to about 100 μm, about 100 μm to about 250 μm, about 250 μm to about 500 μm, about 500 μm (0.5 mm) to about 1000 μm (1 mm), about 1 mm to about 2 mm, about 2 mm to about 5 mm, about 5 mm to about 10 mm (1 cm), about 1 cm to about 2 cm, about 2 cm to about 5 cm, about 15 μm, about 35 μm, about 55 μm, about 75 μm, about 120 μm, about 150 μm, about 300 μm, about 1.5 mm, about 3.5 mm, about 7.5 mm, about 1.2 cm, about 2.5 cm, about 3.5 cm or about 4.5 cm.

The moulds are to be made of a material that allow for accurate and uniform production of the wells, as well as to allow for cells in cell culture to grow in these wells, as an example. Not all surfaces or materials available are suitable for this purpose, thus cell culture grade and medical grade components are chosen as materials from which the moulds were made. In one example, the mould is made of an elastomeric polymer. In another example, the elastomeric polymer is selected from, but not limited to, agarose, polyethylene glycol gels, poly(2-hydroxyethyl methacrylate) gels (or poly (HEMA) gels), silicon rubbers such as polydimethylsiloxane (PDMS), urethane rubbers and ethylene-vinyl acetate (EVA) rubbers. In another example, the elastomeric polymer is polydimethylsiloxane (PDMS).

The moulds are to be populated with the cells or the plurality of cells in order to make the self-supporting cellular constructs. In order to do so, cells are placed into the moulds. A person skilled in the art would be able of performing this task using standard procedures known in cell culture. For example, the plurality of cells can be pipetted directly into the mould or the wells within the mould, which is placed, for example, into a multi-welled cell culture dish containing the respective cell culture growth media. In one example, the plurality of cells is condensed into the mould by centrifugation, such that the cells form the self-supporting cellular construct. In cases where more than one self-supporting cellular structure is needed, a plurality of self-supporting cellular structures are made simultaneously by using a mould that has more than one well. Such a multi-welled mould enables rapid and uniform production of the cellular constructs as described herein. In another example, the mould is a multi-welled mould to thereby obtain a plurality of self-supporting cellular structures attached to the removable material.

In one example, the present invention refers to a method of making or producing a self-supporting cellular construct having a continuous channel within its central cavity, the method comprises the steps of providing a mould with a central opening, wherein the mould encloses a volume around the central opening (hole) capable of housing a plurality of cells. A plurality of cells is inserted, in a cell culture medium, into the mould. The cells are allowed to grow and form a self-supporting cellular construct within the mould. The self-supporting cellular construct, having a central opening and an outer dimension of the mould, is removed from the mould. The afore-mentioned steps are repeated to obtain a plurality of self-supporting cellular construct having a central opening. Each of the plurality of self-supporting cellular constructs, having a central opening in a series adjacent to one another such, is placed so that the central opening of each of the self-supporting cellular constructs having a central opening is aligned to one another to thereby form a continuous channel within its central cavity.

In another example, the present invention refers to a method of making/producing a self-supporting cellular construct, the method comprises the steps of providing a mould of a desired shape, wherein the mould comprises an area capable of housing a plurality of cells and a removable material within the mould. A plurality of cells is inserted, in a cell culture medium, into the mould. A removable material is inserted before or after the preceding step. The cells are allowed to grow and form a self-supporting cellular structure surrounding the removable material within the mould, and the self-supporting cellular structure is removed from the mould to thereby form a self-supporting cellular construct.

The resulting size of the self-supporting cellular construct made using the method as described herein differs according to the intended downstream function of the simple or complex self-supporting cellular construct. In one example, the method is as described herein, further repeating the steps as described herein to obtain a plurality of self-supporting cellular structures on a plurality of removable materials.

This plurality of self-supporting cellular structures on a plurality of removable materials can then be used to assemble larger and/or more complex constructs. These larger and/or more complex constructs can be assembled by methods chosen from, but not limited to, aligning, or weaving, or braiding, or knitting, or knotting to thereby obtain a complex self-supporting cellular construct. The result of these assembly techniques is that the self-supporting cellular construct maintains its structure without the assistance of any external material.

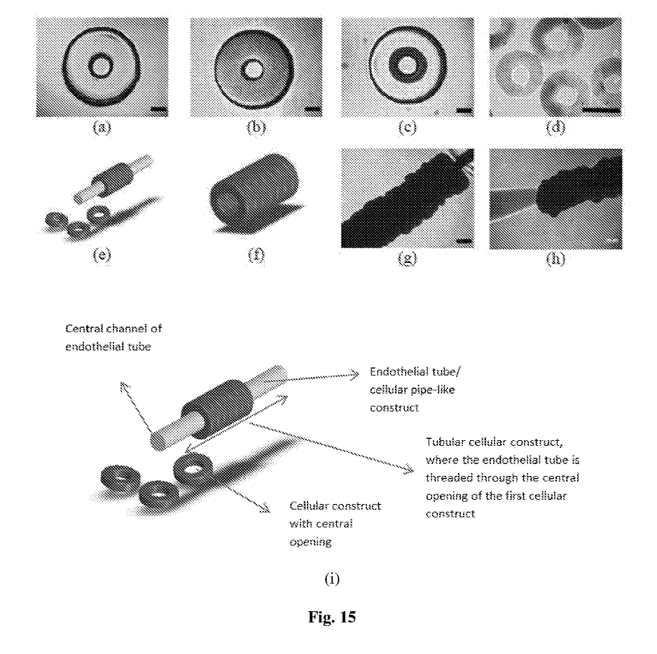

For example, one possible cellular construct made using the present invention was a tube, which represents a series of self-supporting cellular constructs (“rings”) as formed using the method described herein and “threaded” (pushed) onto a central axis, therefore resulting in an aggregation of biological material around the central axis, of which the central axis (that is the removable material) could be removed or retained depending on the downstream application, as illustrated in FIG. 15. This central axis can be retained, for example when more complex structures are being developed and more initial stability is needed. In cases where this central axis is removed, removable material is used, thereby enabling its removal from the finished cellular construct later on before, after or during assembly of a simple or complex cellular construct. Thus, in one example, a removable material is threaded through the central opening of the plurality of self-supporting cellular construct having a central opening, thus forming a series of self-supporting cellular construct. In another example, the present invention refers to the method as described herein, further comprising the step of removing the removable material from the self-supporting cellular structure to thereby form the self-supporting cellular construct with a central channel along the self-supporting cellular construct. In another example, the removable material is removed by physically drawing the removable material away from the plurality of cells formed as a tube. In one example, the present invention refers to the method as described herein, wherein the removable material is removed when the plurality of self-supporting cellular construct has assembled and fused to one another, forming a continuous channel within its central cavity. In another example, the removable material is removed by physically drawing the removable material away from the plurality of self-supporting cellular constructs formed into a tube.

The shape of the removable material must also be taken into consideration as this will dictate the resulting cellular construct and its applicability later on in downstream applications. In the example of the formation of a tube, in order to make a tube, a person skilled in the art would understand that the removable material must therefore take the shape of a long, cylindrical (that is tubular) shape in order to allow the formation of a self-supporting cellular construct having a central opening. In one example, the cellular construct has substantially circular or elliptical cross section. It is also understood that the size of the resulting structures is dictated by the size of the removable material used. In cases where the self-supporting cellular construct is a ring, for example, the form and shape of the removable material, and thus the hole of the self-supporting cellular construct should be the same. That is to say, if the hole of the self-supporting cellular construct (in this case a ring) is round in shape, then the shape of the removable material on which the self-supporting cellular construct is to be threaded should be round. That does not mean that if the shape of the removable material were square that it would not be possible to thread the self-supporting cellular constructs with round holes onto the removable material. The malleability of cells in cell culture would allow such threading. However, more cellular material is needed, as such an imperfect fit between the hole and removable material is expected to result in frictional damage. Therefore, the removable material is provided, for example and not limited to, in the form of a needle (curved, bent or straight), a suture, thread, surgically acceptable wire and the like.

Also, the size of the hole within the self-supporting cellular construct and the removable material to be used should correspond. This means that the size of the removable material is either the same size, or smaller than the size of the hole through which the removable material is to be threaded. The size of the ring can be depicted, for example, as part of the total diameter of the cellular O ring, or as the inner diameter of the cellular O ring. Therefore, the size of the cellular O ring is of about 50 μm to about 100 μm, about 100 μm to about 250 μm, about 250 μm to about 500 μm, about 500 μm (0.5 mm) to about 1000 μm (1 mm), about 1 mm to about 2 mm, about 2 mm to about 5 mm, about 5 mm to about 10 mm (1 cm), about 1 cm to about 2 cm, about 2 cm to about 5 cm, about 55 μm, about 75 μm, about 120 μm, about 150 μm, about 300 μm, about 1.5 mm, about 3.5 mm, about 7.5 mm, about 1.2 cm, about 2.5 cm, about 3.5 cm or about 4.5 cm in diameter. Also, the thickness of the ring, that is the wall thickness of the cellular O ring that is the distance between the outer and the inner wall of the cellular O ring and is, but not limited to, about 10 μm to about 50 μm, about 50 μm to about 100 μm, about 100 μm to about 250 μm, about 250 μm to about 500 μm, about 15 μm, about 35 μm, about 55 μm, about 75 μm, about 120 μm, about 150 μm, about 300 μm in thickness. In one example, the cellular construct forms a cellular O ring with an inner diameter of 5 cm, and wall thickness of 500 μm.

In order to be able to thread or remove the central axis (that is the removable material) from the central cavity of the self-supporting cellular construct, it is advantageous that the removable material does not in any way stick to the central cavity of the cellular construct. Such friction would cause damage to the resulting construct and would undermine the stability of the cellular construct, for example making it prone to leaking, tearing or breaking in use later on. These non-stick agents are used to coat any surface, on which the non-stick, low friction effect is to be seen. Such a non-sticking agent is selected from, but is not limited to, polytetrafluoroethylene (PTFE, Teflon), fluorinated ethylene propylene (FEP), anodized aluminium, ceramic, silicone, enamel, nylon, Whitford Xylan, polyether ether ketone (PEEK), ethylene chlorotrifluoroethylene (ECTFE, Halar), polyvinylidene fluoride, or polyvinylidene difluoride (PVDF, Hylar, Kynar) and perfluoroalkoxy alkane (PFA). In one example, the removable material is a metal needle or a stick coated with non-sticking agent such as polytetrafluoroethylene (PTFE, Teflon).

Another method of removing the removable material from the central opening of the self-supporting cellular construct is if the removable material is biodegradable, that is, the suture is made of material that is readily absorbed into the body over time. This biodegradability would allow the removal of the removable material without damaging the cellular construct or any other construct enclosing it. In one example, the removable material is removed by biodegradation. Dissolvable fibre may also be used in place of the sutures to facilitate removal. An example would be, but is not limited to, a calcium alginate fibre that could be removed by dissolution with sodium citrate buffer. Further examples of dissolvable materials are, but are not limited to, polyglycolic acid, polylactic acid, polydioxanone, and caprolactone.

As an example, a biodegradable suture is a useful delivery tool to deliver the cellular construct into a subject during transplantation as it helps to localise the transplanted cells to the treatment site. A suture also enables better handling of the construct by surgeons, researchers, or any other persons skilled in the art. Therefore, in one example, the removable material is a biodegradable suture.

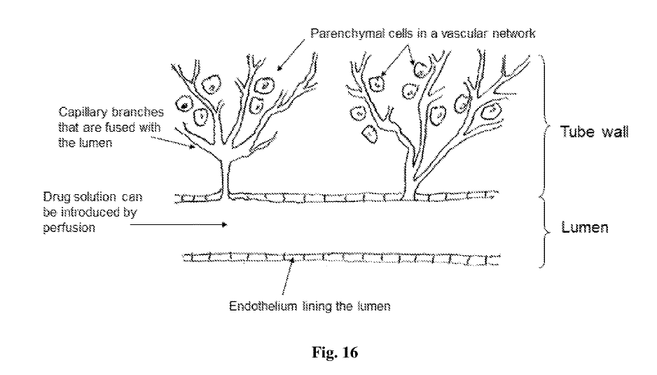

The method as described herein is used to produce simple structures, such as tubes and spheres or more complex cellular constructs that mimic biological counterparts, for example, epithelial linings, the blood-brain-barrier, capillary blood vessels, arteries, veins, kidney cells and hair shafts. FIG. 16 shows an example of a vascular network that may be produced using the method as described herein. Complex cellular constructs are also designed and made to mimic native drug uptake scenarios in order to observe, for example, drug uptake or metabolism through different epithelial cells, or filtration in the kidneys. In one example, the method is as described herein, wherein the plurality of cells mimics native drug uptake scenario. In another example, the plurality of cells is selected from the group consisting of endothelial cells, kidney tubule cells (which are useful in studying the filtration within kidney) and epithelial cells such as intestinal epithelial cells and stomach epithelial cells (which are useful in studying drug uptake kinetics through the epithelium of these organs). In a further example, the plurality of cells further comprises parenchymal cells. These parenchymal cells enable the study of drug uptake kinetics through the endothelium, for example the fenestrated endothelium in the liver and the blood-brain barrier endothelium in the brain. In yet another example, the cellular construct has a continuous channel within its central cavity mimicking a normal size human aortic artery (i.e. largest blood vessel).

The plurality of cells used in the method described herein refers to the use of more than one cell. This can also mean that of the cell population used, single cell types, as well as combinations of different cell types are used to make the self-supporting cellular constructs as described herein. The use of one or more cell types in the assembly of cellular constructs depends on the intended function of that particular part of the construct. Therefore, in one example, the plurality of cells comprises a single type of cell. In another example, the plurality of cells comprises two or more types of cells. In yet another example, the plurality of cells comprises two types of cells.

For example, a plurality of cells made up of hair follicle cells, such as keratinocytes and dermal papilla cells are used in making a cellular construct that mimics a hair shaft and its follicle. When these hair follicle cells are implanted subcutaneously in certain areas, formation of hair shaft can take place. A guide such as a suture is then needed for growth of the hair shaft out of the epidermis. Thus, the tissue which is to be mimicked dictates the structure and the cell types used in the assembly of the resulting self-supporting cellular construct. Therefore, in one example, the plurality of cells mimics hair growth wherein the removable material is a guide for the growth of the hair shaft. In another example, the plurality of cells is selected from, but not limited to endothelial cells (such as HUVEC), fibroblasts, chondrocytes, osteoblasts, hepatocarcinoma cells (such as huh7 cells), human embryonic stem cells (hESCs), human induced pluripotent stem cells (hiPSCs), human mesenchymal stem cells (hMSCs), breast carcinoma cells, muscle cells, kidney cells, pancreatic cells, cardiac cells, liver cells, neuronal cells and hair follicle cells; liver carcinoma cell lines (such as HEPG2 and HUH7), breast cells (such as breast carcinoma cell line MCF7); parenchymal cells; mesenchymal stem cells (hMSC), endothelial cells, hepatocytes, chondrocytes, sarcoma cells, astrocytes, glial cells, podocytes, liver sinusoidal endothelial cells, myoblasts (such as C2C12 mouse myoblast cell lines), progenitor cells derived from pluripotent stem cells and combinations thereof.

Combinations of cells are also used according to the present invention. Using a multitude of cell types, each cell type having their own set of characteristics and varying stability and support, enables fine tuning of different aspects of the resulting complex construct, for example the aspect of rigidity, elasticity, malleability, torque, torsion, stability and variability. Therefore, in one example, the plurality of cells comprises a single type of cells. In another example, the plurality of cells comprises at least two types of cells. In yet another example, the plurality of cells further comprises parenchymal cells. In a further example, the plurality of cell comprises at least one cell type selected from the group consisting of human umbilical vascular endothelial cells (HUVEC), human coronary artery smooth muscle cells (CASMC), human mesenchymal stem cells (hMSC) and hepatocarcinoma cells. In another example, the plurality of cell comprises human umbilical vascular endothelial cells (HUVEC) and any one of human mesenchymal stem cells (hMSC) or human coronary artery smooth muscle cells (CASMC). In one example, the cell is selected from cell types consisting of, but not limited to, endothelial cells, fibroblasts, chondrocytes, osteoblasts, hepatocarcinoma cells, huh7 cells, human embryonic stem cells (hESCs), human induced pluripotent stem cells (hiPSCs), human mesenchymal stem cells (hMSCs), breast carcinoma cells, muscle cells, kidney cells, pancreatic cells, cardiac cells, liver cells, neuronal cells and hair follicle cells.

For example, using cell types known to form more rigid structures (for example, capillary walls) can be used to strengthen a cellular structure, whereas the use of cell types known to from more malleable structures can be used to give a resulting cellular structure more flexibility. Another factor that impacts the physical characteristics of a resulting cellular construct is the ratio between the number of cells used when using a multitude of cell types. For example, in a situation where two cell types are provided, the ratio between these cells can be selected from a ratio of about 1 to 9: about 1, or about 1: about 1, about 2: about 1, about 3: about 1, about 4: about 1, about 5: about 1, about 6: about 1, about 7: about 1, about 8: about 1 or about 9: about 1 of the cell types. In one example, this ratio is a ratio between one cell type that forms self-supporting structure to the other cell type that does not form self-supporting structure. The choice of the ratio would ultimately depend on the intended function of the resulting cellular construct. More importantly, the choice in ratio takes into consideration the required stability of the entire cellular construct, thereby preventing disintegration of the cellular construct before completion of its intended purpose. In one example, the plurality of cell comprising human umbilical vascular endothelial cells (HUVEC) and human mesenchymal stem cells (hMSC) are provided in a ratio that would allow the self-supporting structure to remain stable and not disintegrate. In another example, the ratio of human umbilical vascular endothelial cells (HUVEC) to human mesenchymal stem cells (hMSC) is of about 4: about 1, about 5: about 1, about 6: about 1, about 7: about 1 or about 8: about 1.

Furthermore, some of these pluralities of cells can be capable of forming aggregates, where other pluralities of cells are not capable of aggregates. In one example, the cells are capable of forming aggregates. It is possible to choose a cell type based on their capability to form aggregates or not, based on the intended use and characteristics required of the resulting self-supporting cellular construct. In one example, one cell type is capable of forming cell aggregates and the other cell type is not capable of forming cell aggregates by itself. In another example, the cell type that is capable of forming cell aggregates is selected from the group consisting of mesenchymal stem cells (hMSC), endothelial cells, hepatocytes, chondrocytes, and myoblasts (such as, for example, C2C12 mouse myoblast cell lines). In yet another example, the cell type that is not capable of forming cell aggregates is selected from the group consisting of liver carcinoma cell lines (for example, HEPG2 and HUH7), breast cells (for example, breast carcinoma cell line MCF7), and the like.

Some of these pluralities of cells can also be capable of adhering to the surface of, for example, the culture dish. These adherent cells may also be used in the method described in the present disclosure, for example as anchoring cells that are attached to removable material, to enable further cell types to adhere to the removable material that otherwise would not.

In order to be able to better mimic the function and the characteristics of the tissue ex vivo, capillaries and parenchymal cells (or parenchyma) are also cultivated into self-supporting cellular constructs using the methods disclosed herein. This is done by generating cellular rings as building blocks for the more complex cellular structure and can be accomplished by using a ring mould. Therefore, in one example, the method is as disclosed herein, wherein the central opening of the self-supporting cellular construct has an opening at a first end extending towards a second end and wherein the mould has an outer dimension surrounding and is contiguous (connected) with the second end of the central opening.

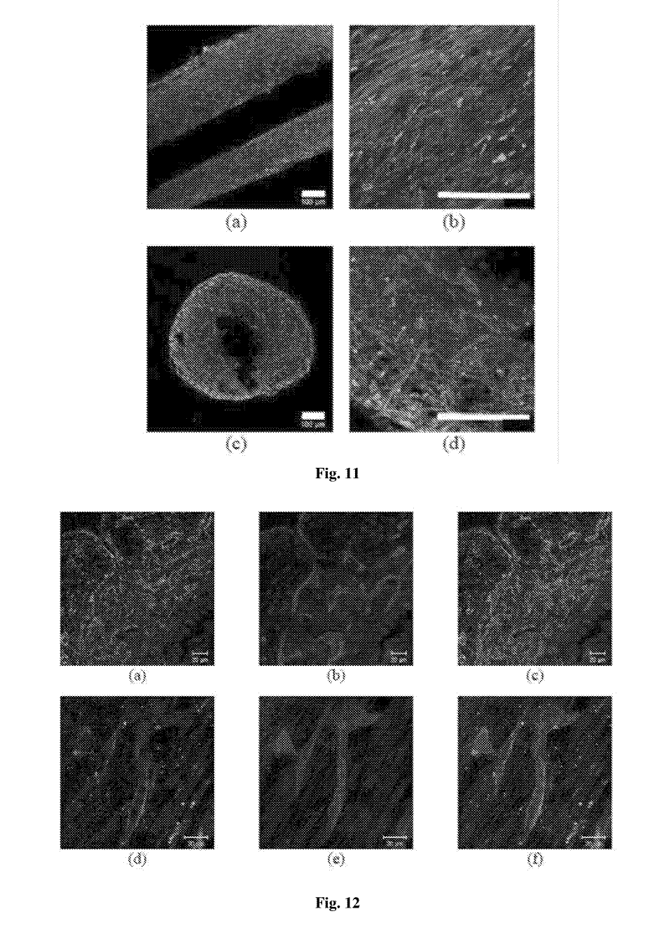

These cellular rings are then assembled to form tubular constructs. For example, a tubular cellular construct made using the methods disclosed herein can then be perfused in vitro, thereby enabling drug testing to be done on cellular constructs using cells to resemble or mimic the function of the vascular network (for example, as shown in FIG. 16). Another use is to provide a vascular axis within the cellular construct for regenerative medicine applications. Vascularization is an important aspect for implantable tissue constructs as it allows cells in the core of these cellular constructs to gain access to oxygen and nutrients. In tissue engineering, vascularization enables the formation of larger organs or organoids. Interactions between endothelium and parenchymal cells are also known to play an important role in organogenesis during embryonic development, as well as maintaining optimal tissue-specific function of the parenchymal cells in adults. In one example, a perfusable vascular network is formed using an endothelial-lined lumen (that is the central channel through the central axis of the tube with is lined with endothelial cells), to which capillary branches are fused. These capillaries are found within the tube walls and are supported and surrounded parenchymal cells, making the entire construct capable of, in this instance, mimicking the vascular network microenvironment ex vivo. Capillaries inherently form within the tube wall, which is characteristic of the endothelial cells when cultured in 3D with peri-vascular cells such as hMSCs and fibroblasts. In another example, this tubulogenesis phenomenon can also take place between the tube wall capillaries and the luminal endothelial cells, fusing the vascular vessels together, for example as shown in FIG. 10. In one example, the cellular construct further comprises at least one capillary that traverse the plurality of cells from the central opening to thereby act as a vascular supply to the plurality of cells in the cellular construct. In another example, the plurality of cells comprises at least one parenchymal cell and the at least one capillary is adjacent to at least one parenchymal cell thereby allowing fluid connection of solution from the central opening to the parenchymal cell. In yet another example, the self-supporting cellular construct further comprises at least one capillary that connects or traverses the self-supporting cellular construct from central channel to an outer dimension of the self-supporting cellular construct. In a further example, the tubular cellular construct further comprises at least one capillary connecting the central channel of the pipe-like cellular construct to the plurality of cells of the cellular construct having a central opening to thereby act as a vascular network connecting the central channel of the pipe-like cellular construct to the plurality of cells in the cellular construct having a central opening.

Some of these pluralities of cells can be further capable of forming extracellular matrix (ECM), where other pluralities of cells are not capable of forming extracellular matrix. Due to its diverse nature and composition, the extracellular matrix can serve many functions, such as providing support for surrounding tissue, segregating tissues from one another, and regulating intercellular communication. The extracellular matrix plays an important role in regulating a cell's dynamic behaviour. In addition, it sequesters a wide range of cellular growth factors and acts as a local store for them. Changes in physiological conditions are known to trigger protease activities, causing local release of such stores. This in turn allows the rapid and local growth factor-mediated activation of cellular functions without requiring de novo synthesis of growth factors. The formation of the extracellular matrix is essential for processes like growth, wound healing, and fibrosis. An understanding of the structure of the extracellular matrix structure and composition also helps in comprehending the complex dynamics of tumour invasion and metastasis in cancer biology as metastasis often involves the destruction of extracellular matrix by enzymes such as serine proteases, threonine proteases, and matrix metalloproteinases. The stiffness and elasticity of the extracellular matrix has important implications in cell migration, gene expression, and differentiation. Therefore, in one example, the cells are capable of forming self-supporting structure, such as extracellular matrix. It is possible to choose a cell type based on their capability to form the extracellular matrix or not, based on the intended use and characteristics required of the resulting self-supporting cellular construct.

As mentioned previously, the possibility of causing an immune reaction when or after implantation is an issue, even when initially, such scaffolds are covered in biological material (for example cultivated cells). After introduction into the host, the cells, for example of a self-supporting cellular construct may migrate away and or around the area of implantation, thereby causing adverse effects and an immune reaction. This type of immune reaction, also known as “foreign body response”, can cause many adverse reactions in the host. The present disclosure provides a method, as described herein, whereby self-supporting cellular constructs are made without the need for further foreign material, or in the case where a complex cellular structure requires initial stabilisation, which is to be removed later on, removable material is used for initial stability, as described herein. Therefore, in one example, a material-free cellular construct is obtained by the method as disclosed herein. In another example, the present disclosure refers to a material-free tubular cellular construct as obtained by the method as disclosed herein.

In one example, the present disclosure discloses the material-free cellular construct as described herein or tubular cellular construct, as described herein, for testing chemical agent. The chemical agent can be tested according to the following method, wherein the method of testing chemical agent comprises the steps of providing the cellular construct as described herein. The chemical agent is provided through the channel of the cellular construct and changes to the plurality of cells of the cellular constructs is observed. In another example, the cellular constructs as described herein are the pipe-like cellular construct and/or cellular construct having a central opening.

The observed effect of the chemical agent on the cells comprised within the self-supporting cellular construct can be seen in different ways. Changes in cell morphology, cell genomics (for example gene expression), cell proteomics (for example protein expression) can give rise to changes that effect the cell microenvironment. The self-supporting cellular constructs can be useful infection models, enabling the observation of the effect of infectious agents on surrounding tissue, as well as the effect of potential treatments on not only the infection agents, but also on the outlying tissues. Another possible application is the study of metastasis in cancer cells, enabling the observation of for example, the migration of the cells, the required vascularization and the leakiness of the vascular system, all of which are important factors in metastasis formation and spreading. In one example, the changes are differential expression of peptide or gene expression as compared to a non-treated control. In another example, the method as described herein is an in vitro method.

The chemical agent that is being observed is, but is not limited to, drugs, chemicals, pharmaceutical compositions, pharmaceutical compounds, epigenetic modulators, genetic modulators, oligonucleotides, peptides, nucleic acids, polypeptides, siRNA, shRNA, iRNA, intercalators, transcription factors, antibiotics and antibodies. In one example, the material-free cellular construct, the tubular cellular construct, or the methods are as described herein, wherein the chemical agent is a pharmaceutical compound. The chemical agent may further be provided in the form of, but not limited to, a drug delivery vehicle, e.g. liposomes, nanoparticles, microspheres and the like.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### Experimental Section

### Methods

Cell Seeding into Micro-Well Mould

The PDMS micro-well moulds were placed at the bottom of a well in a 24-well multi-well cell culture plate. 500 μl of culture medium was added to the well, and the culture plate was centrifuged at 400 g for 3 minutes to remove air bubbles from the micro-wells. Next, 500 μl of cell suspension was added to the well, and the culture plate was centrifuged at 100 g for 3 minutes. At the end of centrifugation, cells were packed into the micro-wells and ready for subsequent culture. Similar processes were used to pack cells into the micro-channel moulds to form cellular tubes. All cell culture methods here are performed according to protocols and knowledge known in the art of cell culture.

Integrating Suture into the Mould

A surgical scalpel was used to cut slits into the moulds, for example PDMS moulds. The slits made to the moulds were made where the sutures were to be placed, which depends on the different mould designs. For example in FIG. 7a, slits are made at the two ends of the rectangular slot so that the suture could fit into the slots. For moulds to be used with the cellular o rings, sutures were not fitted into the mould, as these cellular rings were first formed, washed out from the mould and subsequently, threaded onto a suture. Next, sutures were inserted into these slits. Both ends of the suture were pulled to ensure that the suture remained taut in the slits. If required, the suture can also be anchored to the sides of the mould to ensure tautness. Cells were seeded using the procedures described in the previous paragraph. The resulting structures were later removed from the moulds together with the sutures. For example, as cells do not attach to PDMS, the cellular structures were removed by gently rinsing with phosphate-buffered saline (PBS) for those not attached on sutures, or the sutures can be removed gently from the mould with the cellular structures still attached to the sutures.

### Formation of 3D Cellular Structures

A scalable method to form 3D cellular units for assembly into hierarchical structures has been developed without the use of hydrogel or scaffold materials. Cellular units are first formed in predetermined shapes using polydimethylsiloxane (PDMS) moulds and are subsequently assembled into higher order structures to mimic native tissues.

In the example shown in FIG. 1, cellular spheroids are assembled around a cellular tube that was condensed on a biodegradable suture. The cellular spheroids consist of a mixture of hepatocarcinoma cells (hepG2) and endothelial cells (FIG. 1a-c) while the cellular tube consists of endothelial cells (FIG. 1d-f). The assembled structure provides a vascular axis made of endothelial cells, surrounded by hepatocarcinoma cell aggregates interspersed with vascular capillaries (FIG. 1g-i). This structure mimics the hierarchical vascular network within a liver tissue, and is useful for making tissue structures for regenerative medicine and drug testing applications.

### Formation of Cellular Spheroids

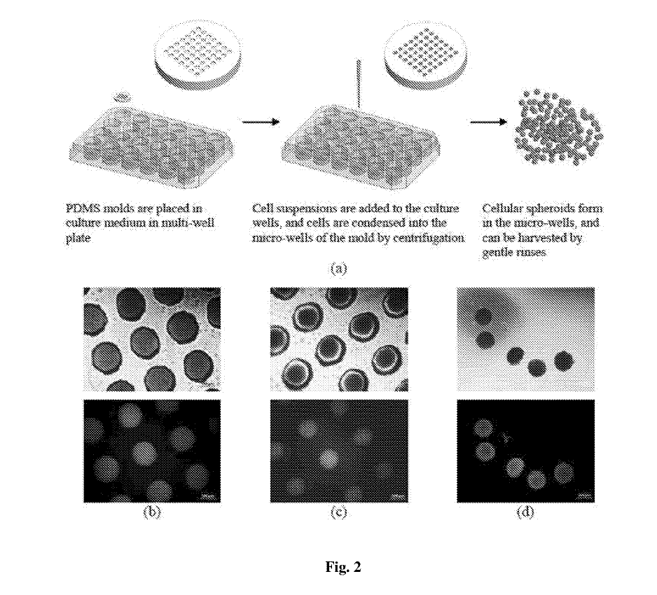

By using a PDMS mould with circular micro-wells, it is possible to create cellular spheroids of uniform size within a short time. As shown in FIG. 2, cell suspensions are added to moulds that are placed in the multi-well culture plate. Subsequent centrifugation causes the cells to settle into the micro-wells. After 1 day in culture, cellular spheroids of uniform size could be obtained.

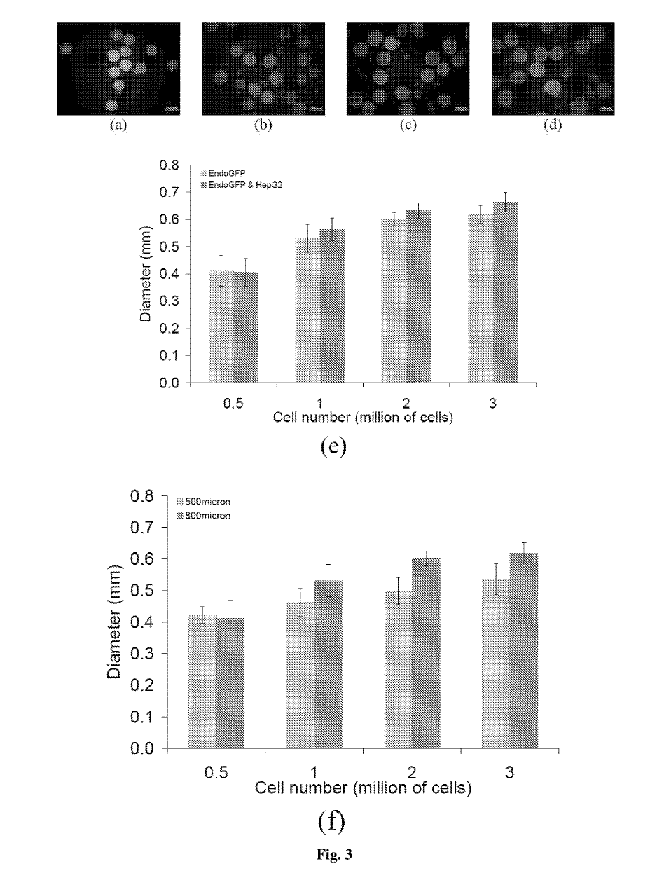

The size of the cellular spheroids can be changed by varying the cell number seeded onto the PDMS mould. A mixture of cell types can also be used to form cellular spheroids. FIG. 3 shows that the size of the cellular spheroids increases with cell number.

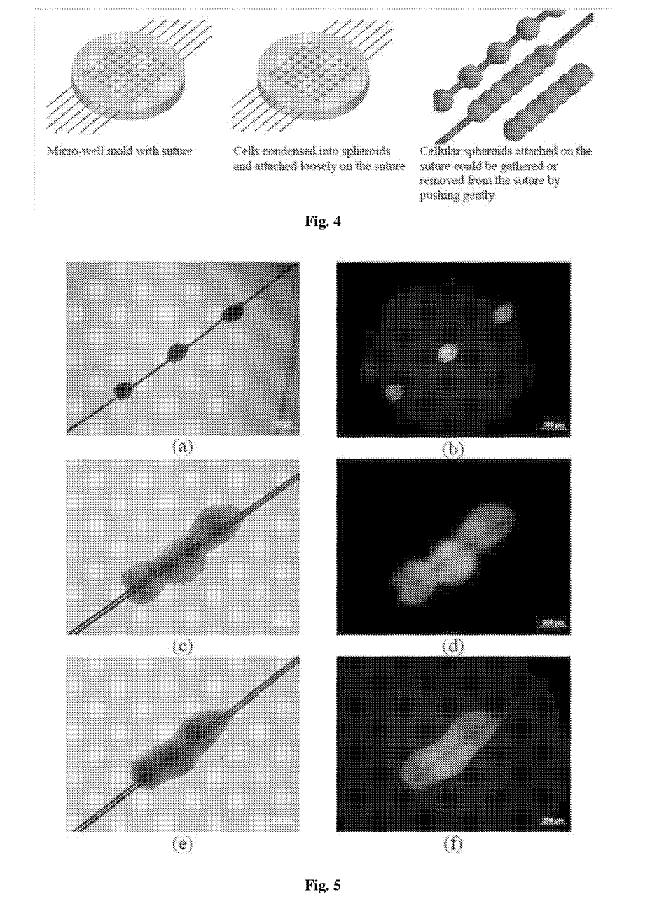

A variant of the technique shown in FIG. 2 is the integration of sutures into the mould (FIG. 4). In doing so, it was possible to obtain cellular spheroids that are attached loosely to the suture. These cellular spheroids can be pushed gently along the suture, by using a pair of forceps or sliding the spheroids on the surface of the culture medium, such that they come into contact with other spheroids. Neighbouring spheroids fuse to form into a string of spheroids after subsequent culture.

Using this technique, cell patterning along the suture is achieved. As shown in FIG. 5, spheroids of different cell types can be patterned with different configurations on the suture (FIGS. 5(a) and (b)). The cellular spheroids can be brought into contact (FIGS. 5(c) and (d)) and allowed to interact and fused into one patterned cellular unit (FIGS. 5(e) and (f)).

### Formation of Cellular Tubes

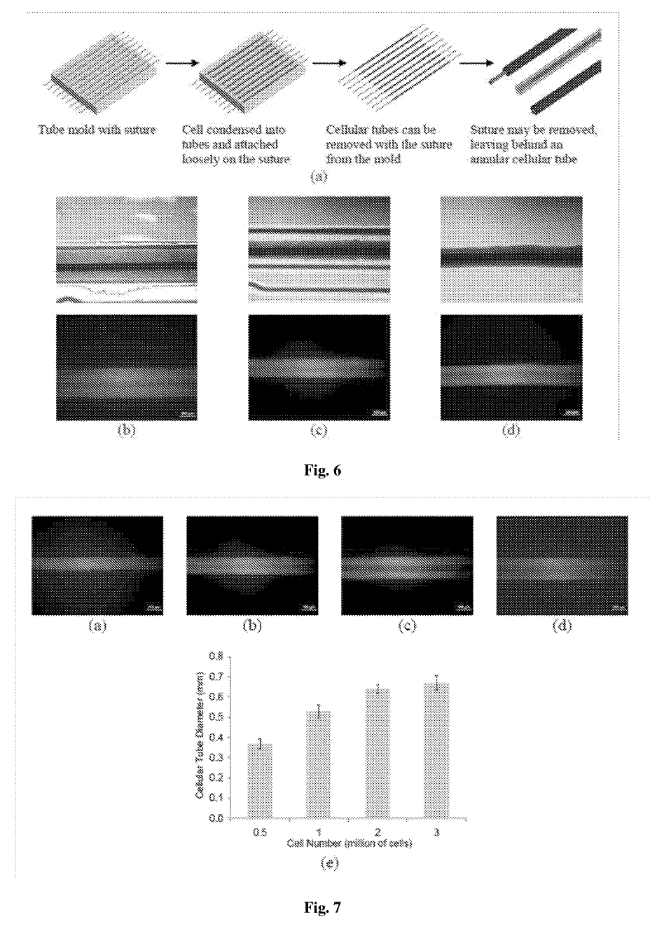

This technique also allows the formation of cellular tube on a suture, which can be used to provide a vascular axis for subsequent assembly. By integrating suture into the mould design, we are able to obtain cellular structures which are condensed onto the suture (FIG. 6). These cellular tubes are harvested by removing them together with the suture from the PDMS mould. Subsequent removal or degradation of the suture results in an annular tube for perfusion and conditioning of the assembled cellular structure.

The diameter of the cellular tube increased with cell number as more cells aggregated around the suture (FIG. 7). For a channel of size 2.5 cm by 0.1 cm, the optimal cell numbers were 1 and 2 million cells, respectively. 0.5 million cells yielded discontinuous cellular tubes, while 3 million cells resulted in an overflow of cells outside the channel.

Formation of Cellular Tube with Primary Cells

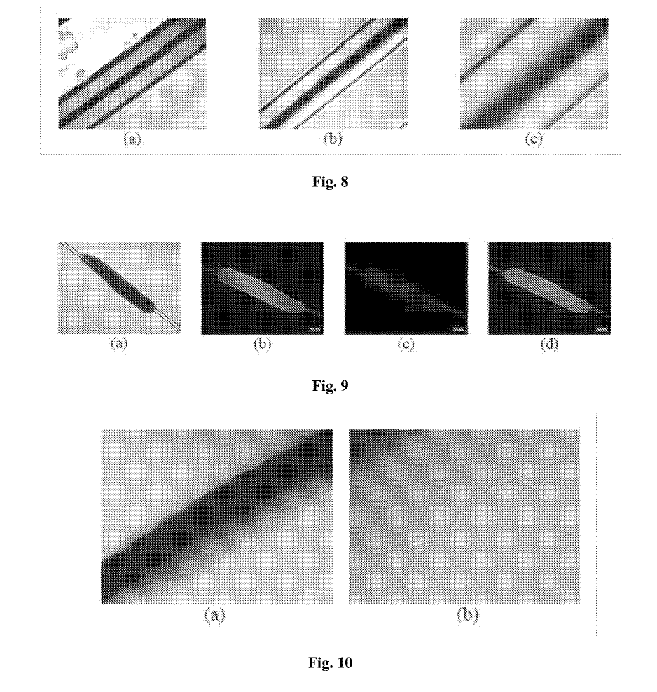

It is also important that the cellular tubes can be formed with primary human cells. To demonstrate this, human umbilical vascular endothelial cells (HUVEC) are used. However, when HUVEC were seeded, the cells did not aggregate around the suture. The problem was solved by seeding HUVEC with another cell type such as human mesenchymal stem cells (hMSC) or human coronary artery smooth muscle cells (CASMC). Cellular tubes were obtained when HUVEC were mixed with hMSC at a ratio of 4:1 (FIG. 8). Ratios of 1:1, 6:1 and 8:1 yielded similar results. However, cellular tubes were not formed for ratios 10:1 and beyond.

These HUVEC cellular tubes are cultured for 7 days to allow the cells to form their own extracellular matrices (ECM). Good cell viability was observed at day 7 (FIG. 9). The cellular tube remained intact on the suture after 7 days.

The HUVEC tubes are also cultured for 3 days and embedded in collagen gel for tubulogenesis assay. HUVEC within the cellular tubes were able to migrate into the gel and form stable endothelial tubules at day 7 as shown in FIG. 10.

The cellular structures do not contain any foreign materials, with the exception of a biodegradable surgical suture that can be resorbed in the body. Without the structural support from other materials, it is important that the cells within the structures are able to synthesize their own extracellular matrix in vitro. To demonstrate this, the HUVEC tubes are cultured for 7 days and stained for the extracellular matrix components collagen I, collagen IV and laminin. The former is an important structural protein found in extracellular matrix while the remaining proteins are components of the basement membrane found in endothelial tubules. From FIG. 11, it was observed that collagen I was present in abundance in the cellular tubes. A CD31 positive endothelial network was also observed within the collagen I rich structure. In addition, the presence of basement membrane components, collagen IV and laminin was observed, adjacent to CD31 positive endothelial tubules formed within the HUVEC tube (FIG. 12).

Cellular Tube Combined with other Hydrogel Encapsulation Techniques

To mimic the structure of an artery, the cellular tube are wrapped circumferentially by smooth muscle cells (SMC) laden interfacial polyelectrolyte (IPC) fibre hydrogel. The formed construct was cultured to allow the smooth muscle cells to align in the collagen-containing IPC fibres (FIG. 13).

### Assembly of Cellular Units

The use of biodegradable suture in this technique facilitates assembly of cellular units. Sutures can be aligned, weaved, braided, knitted and knotted, thereby providing a way to assemble the cellular units. An example of an assembled structure by aligning and packing sutures together is shown in FIG. 14. The strings of cellular spheroids are arranged around a cellular tube, which act as a vascular axis to facilitate construction of thick tissue structure.

If necessary, the sutures may be removed from the cellular structures by biodegradation in vitro or in vivo, or by sliding them from the cellular units after the cells have deposited their own extracellular matrices. Alternatively, cellular rings are formed on PDMS moulds and subsequently assembled on a rod as shown in FIG. 11. The cellular rings fused after culturing for 3 days and the resultant cellular tube is removed from the rod. Perfusion can be carried out through the lumen of the cellular tube, which remained intact as shown in FIG. 15.

This technique allows the formation of cellular units with controllable shapes and sizes, which can then be assembled to more complex structures with defined architecture mimicking native tissues. This allows the application of this technique to various tissue engineering applications, for example:

### Recapitulating Tissue Structures

This technique allows the mimicry of native tissue structures, which can be used to further understand and elucidate important functions. For example, articular cartilage tissue consists of different zones which contribute to its load-bearing ability. In our technique, sutures can be arranged to form layers of cellular units which can consist of cartilage cells of a particular zone. These layers can be stacked subsequently to form a zonal tissue. Hair follicle structure can be mimicked using this technique by forming a dermal papilla (DP) cellular spheroid on a suture, and subsequently allowing epithelial cells to condense around the dermal papilla spheroids. Nerve tissue can be engineered by aligning neurons along the suture and surrounding them with insulating cells such as Schwann cells or oligodendrocytes. Large diameter blood vessels can be formed by aligning smooth muscle cells circumferentially around endothelial tubes as shown in FIG. 9. The ‘inside-out’ approach ensures formation of a complete endothelialized lumen, which is an existing challenge for techniques that requires seeding of endothelial cells in pre-formed tubular structures.

Vascularization for Tissues (e.g. Liver)

One of the major challenges in engineering thick tissues is ensuring vascularization in vivo. The method disclosed herein allows the micro-patterning of cellular units around an endothelial tube, thereby ensuring that the cells are within the nutrient diffusion limits in vivo. In addition, a vascular network is created by integrating the capillaries within the cellular spheroids with the endothelial tube. This is applied to the engineering of various tissues, e.g. liver, pancreas, cardiac muscle etc.

### Rapid Formation of Tissue

The method as described herein also allows rapid formation of cellular units and structures, which are used for tissue repair and replacement in trauma cases.

### In Vitro Perfusion Cellular Model for Drug Testing

The assembled tissue structures are attached to a perfusion system in vitro via the endothelial tube after removal of the rod (as shown in FIG. 15). This setup is used for testing of drugs and analysis of the metabolites through the flow system, and can be applied to various cell types (liver, kidney, pancreas).

1. A method of making a self-supporting cellular construct having a continuous channel within its central cavity, wherein the method comprises the steps of:
(a) providing a mould with a central opening, wherein the mould encloses a volume around the central opening (hole) capable of housing a plurality of cells;
(b) inserting a plurality of cells in a cell culture medium into the mould;
(c) allowing the cells to grow and form a self-supporting cellular construct within the mould;
(d) removing the self-supporting cellular construct having a central opening and an outer dimension of the mould from the mould;
(e) repeating a to d to obtain a plurality of self-supporting cellular construct having a central opening;
(f) placing each of the plurality of self-supporting cellular constructs having a central opening in a series adjacent to one another such that the central opening of each of the self-supporting cellular constructs having a central opening is aligned to one another to thereby form a continuous channel within its central cavity.

2. The method of claim 1, wherein a removable material is threaded through the central opening of the plurality of self-supporting cellular construct having a central opening, thus forming a series of self-supporting cellular construct.

3. The method of claim 2, wherein the removable material is removed by physically drawing the removable material away from the plurality of cells formed as a tube.

4. The method of claim 3, wherein the removable material is a metal needle or a stick, or wherein the removable material is coated with non-sticking agent.

5.-7. (canceled)

8. The method of claim 1, wherein the removable material is removed by biodegradation.

9. The method of claim 8, wherein the removable material is a biodegradable suture.

10. The method of claim 1, wherein the removable material is removed when the plurality of self-supporting cellular construct has assembled and fused to one another, forming a continuous channel within its central cavity.

11. (canceled)

12. (canceled)

13. The method of claim 1, wherein the plurality of cells comprises a single type of cell.

14. The method of claim 13, wherein the cell is selected from the group of cell types consisting of endothelial cells, fibroblasts, chondrocytes, osteoblasts, hepatocarcinoma cells, human embryonic stem cells (hESCs), human induced pluripotent stem cells (hiPSCs), human mesenchymal stem cells (hMSCs), breast carcinoma cells, muscle cells, kidney cells, pancreatic cells, cardiac cells, liver cells, neuronal cells and hair follicle cells.

15. The method of claim 1, wherein the plurality of cells comprises at least two types of cells.

16. The method of claim 15, wherein one cell type is capable of forming cell aggregates and the other cell type is not capable of forming cell aggregates by itself

17. The method of claim 15, wherein the two cell types are provided with a ratio of about 1: about 1, about 2: about 1, about 3: about 1, about 4: about 1, about 5: about 1, about 6: about 1, about 7: about 1, or about 8: about 1 of the cell type that forms self-supporting structure to the other cell type that does not form self-supporting structure.

18. The method of claim 1, wherein the cells are capable of forming self-supporting structure, such as extracellular matrix.

19. The method of claim 16, wherein the cell type that is capable of forming cell aggregates is selected from the group consisting of mesenchymal stem cells (hMSC), endothelial cells, hepatocytes, chondrocytes, and myoblasts.

20. The method of claim 16, wherein the cell type that is not capable of forming cell aggregates is selected from the group consisting of liver carcinoma cell lines, and breast cells.

21. The method of claim 1, wherein the plurality of cells further comprises parenchymal cells.

22. The method of claim 21, wherein the plurality of cell comprises at least one cell type selected from the group consisting of human umbilical vascular endothelial cells (HUVEC), human coronary artery smooth muscle cells (CASMC), human mesenchymal stem cells (hMSC) and hepatocarcinoma cells.

23. The method of claim 22, wherein the plurality of cell comprises human umbilical vascular endothelial cells (HUVEC) and any one of human mesenchymal stem cells (hMSC) or human coronary artery smooth muscle cells (CASMC).

24. The method of claim 23, wherein the plurality of cell comprising human umbilical vascular endothelial cells (HUVEC) and human mesenchymal stem cells (hMSC) are provided in a ratio that would allow the self-supporting structure to remain stable and do not disintegrate.

25. The method of claim 24, wherein the ratio of human umbilical vascular endothelial cells (HUVEC) to human mesenchymal stem cells (hMSC) is of about 1: about 1, about 4: about 1, about 5: about 1, about 6: about 1, about 7: about 1 or about 8: about 1.

26. The method of claim 1, wherein the cellular construct further comprises at least one capillary that traverses the plurality of cells from the central opening to thereby act as a vascular supply to the plurality of cells in the cellular construct.

27. The method of claim 26, wherein the plurality of cells comprises at least one parenchymal cell and the at least one capillary is adjacent to at least one parenchymal cell thereby allowing fluid connection of solution from the central opening to the parenchymal cell.

28. The method of claim 1, wherein the central opening has an opening at a first end extending towards a second end and wherein the mould has an outer dimension surrounding and is contiguous (connected) with the second end of the central opening.

29. A method of making a self-supporting cellular construct, the method comprises the steps of:
(a) providing a mould of a desired shape, wherein the mould comprises an area capable of housing a plurality of cells and a removable material within the mould;
(b) inserting a plurality of cells in a cell culture medium into the mould;
(c) inserting a removable material before or after (b);
(d) allowing the cells to grow and form a self-supporting cellular structure surrounding the removable material within the mould; and
(e) removing the self-supporting cellular structure from the mould to thereby form a self-supporting cellular construct.

30.-54. (canceled)

55. A method of testing chemical agent comprising the steps of:
(a) providing the cellular construct as claimed in claim 1; and
(b) providing the chemical agent through the channel of the cellular construct and
(c) observing changes to the plurality of cells of the cellular constructs.

56.-58. (canceled)
